(19) United States
(12) Patent Application Publication
(10) Pub. No.: US 2020/0392126 A1
(43) Pub. Date: Dec. 17, 2020

(54) ISOXAZOLYL COMPOUNDS AS RECEPTOR MODULATING COMPOUNDS AND METHODS AND USES THEREOF

(71) Applicant: BIONOMICS LIMITED, Thebarton, South Australia (AU)

(72) Inventors: Justin Anthony RIPPER, Thebarton, South Australia (AU); Patrick BAZZINI, Strasbourg (AU); Jean-Marie CONTRERAS, Westhouse (FR); Nicolas FEICHTER, Illkirch (FR); Christophe MORICE, Widensolen (FR); Laurent SCHAEFFER, Fortschwihr (FR); Cindy STAHL, Siewiller (FR)

(73) Assignee: BIONOMICS LIMITED, Thebarton, South Australia (AU)

(21) Appl. No.: 16/769,757

(22) PCT Filed: Nov. 26, 2018
(86) PCT No.: PCT/AU2018/051258

§ 371 Date: Jun. 4, 2020

(30) Foreign Application Priority Data
Dec. 5, 2017 (AU) 2017904893

(51) Int. Cl. C07D 413/12; C07D 261/10; C07D 413/14

(52) U.S. Cl. CPC C07D 413/12; C07D 413/14; C07D 261/10

(57) ABSTRACT

The present invention relates generally to chemical compounds and methods for their use and preparation. In particular, the invention relates to chemical compounds which are useful in relation to the treatment of diseases, disorders or conditions which would benefit from the modulation of the alpha 7 nicotinic acetylcholine receptor (α7 nAChR). The invention also relates to the use of these compounds in methods of therapy and the manufacture of medicaments as well as compositions containing these compounds.

### FIELD

The present invention relates generally to chemical compounds and methods for their use and preparation. In particular, the invention relates to chemical compounds which are useful in relation to the treatment of diseases, disorders or conditions which would benefit from the modulation of the alpha 7 nicotinic acetylcholine receptor (α7 nAChR). The invention also relates to the use of these compounds in methods of therapy and the manufacture of medicaments as well as compositions containing these compounds.

### BACKGROUND

The α7 nAChRs are rapidly desensitizing cation channels belonging to the cis-loop ligand-gated family. α7 nAChRs play an important role in the regulation of neuronal excitability in different brain regions either by presynaptically modulating neurotransmitter release or by their position on somato-dendritic sites of interneurons and pyramidal cells, where they directly regulate neuronal activity. They are abundantly expressed in the brain, modulate neurotransmitter release and are responsible for direct fast excitatory neurotransmission. At the cellular level, activation of α7 nAChRs can regulate interneuron excitability, modulate the release of excitatory and inhibitory neurotransmitters, and contribute to neuroprotective effects. Positive modulation of α7 nAChRs can enhance hippocampal LTP, and α7 nAChRs are associated with attentional processes and working memory. As a consequence, α7 nAChRs are a therapeutic target for treating cognitive impairment, notably in Alzheimer's disease and schizophrenia (1-4).

However, here are several lines of evidence to suggest that excessive amounts of acetylcholine (ACh) in the brain contribute to anxious and depressed mood states (5). One of the most consistent findings in neuropsychiatry is that patients with depression have dysfunctional neuroendocrine systems possibly resulting from prolonged responses to stress (6,7) and that ACh plays a significant role in mediating neuroendocrine, emotional, and physiological responses to stress. For example, central ACh turnover is increased following stress and ACh facilitates the release of several stress-sensitive neuro-hormones and peptides including corticosterone, adrenocorticotropic hormone (ACTH), and corticotropin releasing factor (CRF). A human imaging study has shown that ACh levels are elevated in patients who are depressed, and remain high in patients who have a history of depression (8). In addition, other clinical and preclinical studies have shown that blockers of cholinergic receptors can induce antidepressant-like responses (9). In these human studies, physostigmine, an inhibitor of acetylcholinesterase, increased depressive symptoms in individuals with or without a history of depression. These observations suggest that hyperactivity of brain cholinergic systems can contribute to the pathophysiology of depression (10).

Studies have shown that α7 nAChRs in both the hippocampus and the amygdala are implicated in the regulation of depression and anxiety. The role of cholinergic signaling in the hippocampus in anxiety- and mood-related behaviors has been investigated in rodents using pharmacological (physostigmine) and molecular genetic techniques (shRNAs targeting AChE) to alter AChE levels or activity in adulthood (11). In addition, direct infusion of physostigmine or shRNAs into the hippocampus decreased hippocampal AChE which correlated with increased anxiety and depression-like behaviors and decreased resilience to repeated stress in a social defeat paradigm. The behavioral changes due to shRNA-mediated knockdown of AChE were rescued by co-infusion of an shRNA-resistant AChE transgene into the hippocampus and reversed by systemic administration of fluoxetine. These data demonstrate that ACh signaling in the hippocampus promotes behaviors related to anxiety and depression and suggest that abnormalities in the cholinergic system may be critical for the etiology of mood disorders.

Similarly, cholinergic signaling in the basolateral amygdala has also been implicated in behaviors related to stress. The basolateral amygdala (BLA) receives dense cholinergic input from the basal forebrain, affecting both normal functions and dysfunctions of the amygdala. Neuronal excitability in the basolateral nucleus of the amygdala (BLA) is particularly relevant to anxiety. In the BLA, α7 nAChRs are present on somatodendritic regions of glutamatergic neurons (12), and on somatic and/or dendritic regions of GABAergic interneurons (13). Pre-clinical studies have implicated both β2 subunit-containing (β2*) and α7 nAChRs in the effects of nicotine in models of anxiety- and depression-like behaviors. Viral-mediated down-regulation of the β2 or α7 nAChR subunit in the amygdala induced robust anxiolytic- and antidepressant-like effects in several mouse behavioral models (14). α7 nAChR subunit knockdown was effective at decreasing anxiety-like behavior, and reversed the effect of increased ACh signaling in a mouse model of depression. These results suggested that stimulation of α7 nAChRs by acetylcholine may mediate the increased depression-like behaviors during the hyper-cholinergic state observed in depressed individuals and in those with anxiety.

In summary, there is considerable evidence in the literature to suggest that selective inhibition of α7 nAChRs has therapeutic potential for the treatment of anxiety, depression and stress-related disorders. The present invention seeks to provide such compounds.

### SUMMARY OF THE INVENTION

According to an aspect, the present invention provides compounds of formula (I) or pharmaceutically acceptable salts, solvates, prodrugs, stereoisomers or tautomers thereof:

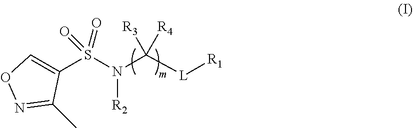

wherein R₁ is selected from optionally substituted heterocyclyl, optionally substituted aryl, optionally substituted 6-membered heteroaryl; and
R₂ is selected from H, optionally substituted alkyl, optionally substituted aryl, optionally substituted cycloalkenyl, optionally substituted cycloalkyl, optionally substituted heteroaryl, optionally substituted heterocyclyl, optionally substituted acyl, or optionally substituted oxyacyl; and
R₃ and R₄ are independently selected from H, hydroxyl, optionally substituted alkyl, optionally substituted alkoxyl, optionally substituted alkenyl, optionally substituted alkynyl, optionally substituted cycloalkyl, optionally substituted heterocyclyl, optionally substituted aryl, optionally substituted heteroaryl, or optionally substituted cycloalkenyl; or
R₃ and R₄ can be linked to form optionally substituted cycloalkyl, optionally substituted heterocyclyl, or optionally substituted cycloalkenyl; and
L is a single bond, N, CH, or a divalent linker selected from CH₂. optionally substituted C₃-C₆ cycloalkylene and optionally substituted C₃-C₆ cycloalkenylene; or
R₂ and L can be linked to form optionally substituted N-containing heterocyclyl where L is N or CH; or
R₂ and R₁ can be linked to form optionally substituted N-containing heterocyclyl where L is CH₂; and
m is an integer selected from 1 to 5,
excluding the following two compounds:

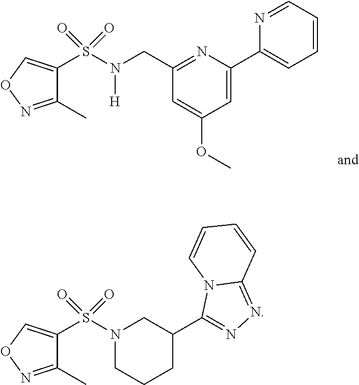 [SMILES: COc1cc(CNS(=O)(=O)c2conc2C)nc(-c2ccccn2)c1 ; Cc1nocc1S(=O)(=O)N1CCCC(c2nnc3ccccn23)C1]

In another aspect the invention provides methods of medically treating a disease, disorder, or condition which would benefit from modulation of α7nAChR, said method including the step of administering an effective amount of a compound of formula (I) or a pharmaceutically acceptable salt, solvate, prodrug, stereoisomer or tautomer thereof.

In another aspect the invention provides the use of compounds of formula (I) or pharmaceutically acceptable salts, solvates, prodrugs, stereoisomers or tautomers thereof, in the manufacture of a medicament for treating a disease, disorder or condition which would benefit from modulation of α7 nAChR.

In a further aspect the invention provides the use of compounds of formula (I) or pharmaceutical salts, solvates, prodrugs, stereoisomers or tautomers thereof, for treating a disease, disorder or condition which would benefit from modulation of α7 nAChR.

In relation to the aforementioned methods and uses in certain embodiments the disease, disorder or condition which would benefit from modulation of α7 nAChR is a disease, disorder or condition in which the benefit comes from the negative allosteric modulation of α7 nAChR.

In certain embodiments the disease, disorder or condition in which the benefit comes from the negative allosteric modulation of α7 nAChR is a disease, disorder or condition selected from anxiety, depression, or a stress-related disorder.

In certain embodiments, the invention provides compounds, methods and uses based on compounds of sub-formulae (I) below or pharmaceutically acceptable salts, solvates, prodrugs, stereoisomers or tautomers thereof:

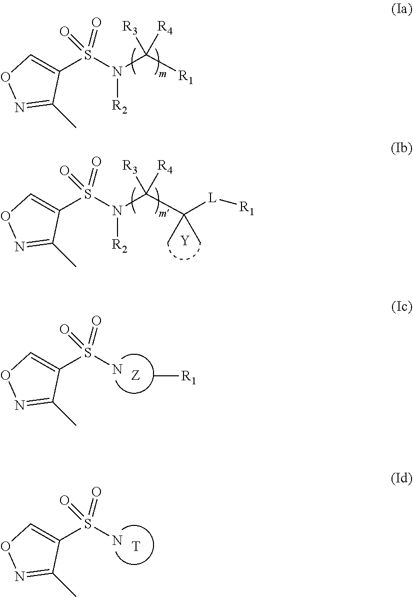

wherein R₁, R₂, R₃, R₄, L and m are as defined above (and the exclusion of the two compounds shown above);
wherein Y is where one R₃ and R₄ is linked together to form optionally substituted cycloalkyl, optionally substituted heterocyclyl, or optionally substituted cycloalkenyl;
wherein Z is where R₂ and L is linked together to form optionally substituted N-containing heterocyclyl where L is N or CH;
wherein T is where R₂ and R₁ is linked together to form optionally substituted N-containing heterocyclyl where L is CH₂; and
m′ is 0 or 1.

### BRIEF DESCRIPTION OF THE FIGURES

FIG. 1—Bar graph illustrating number of entries into lit box vs compound example 3 of various dosages compared to vehicle control.

### DETAILED DESCRIPTION OF THE INVENTION

The present invention is primarily based on the discovery that the compounds of the general formula (I) and sub formula thereof (or pharmaceutically acceptable salts, solvates, prodrugs, stereoisomers or tautomers thereof) have useful properties as selective negative allosteric modulators of alpha 7 nicotinic acetylcholine receptors. Such compounds have significant potential for the treatment of mood disorders such as anxiety, depression and stress related disorders whether they occur alone or as comorbidities with other conditions.

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Alkylene” refers to divalent alkyl groups preferably having from 1 to 10 carbon atoms and more preferably 1 to 6 carbon atoms. Examples of such alkylene groups include methylene (—CH₂—), ethylene (—CH₂CH₂—), and the propylene isomers (e.g., —CH₂CH₂CH₂— and —CH(CH₃)CH₂—), and the like.

“Aryl” refers to an unsaturated aromatic carbocyclic group having a single ring (e.g., phenyl) or multiple condensed rings (e.g., naphthyl or anthryl), preferably having from 6 to 14 carbon atoms. Examples of aryl groups include phenyl, naphthyl and the like.

“Arylene” refers to a divalent aryl group wherein the aryl group is as described above.

“Aryloxy” refers to the group aryl-O- wherein the aryl group is as described above.

“Arylalkyl” refers to -alkylene-aryl groups preferably having from 1 to 10 carbon atoms in the alkylene moiety and from 6 to 10 carbon atoms in the aryl moiety. Such arylalkyl groups are exemplified by benzyl, phenethyl and the like.

“Arylalkoxy” refers to the group arylalkyl-O- wherein the arylalkyl group are as described above. Such arylalkoxy groups are exemplified by benzyloxy and the like.

“Alkoxy” refers to the group alkyl-O- where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Alkenyloxy” refers to the group alkenyl-O- wherein the alkenyl group is as described above.

“Alkenylene” refers to divalent alkenyl groups preferably having from 2 to 8 carbon atoms and more preferably 2 to 6 carbon atoms. Examples include ethenylene (—CH═CH—), and the propenylene isomers (e.g., —CH₂CH═CH— and —C(CH₃)═CH—), and the like.

“Alkynyl” refers to alkynyl groups preferably having from 2 to 10 carbon atoms and more preferably 2 to 6 carbon atoms and having at least 1, and preferably from 1-2, carbon to carbon, triple bonds. Examples of alkynyl groups include ethynyl (—C≡CH), propargyl (—CH₂C≡CH), pent-2-ynyl (—CH₂C≡CCH₂—CH₃), and the like.

“Alkynyloxy” refers to the group alkynyl-O- wherein the alkynyl groups is as described above.

“Alkynylene” refers to the divalent alkynyl groups preferably having from 2 to 8 carbon atoms and more preferably 2 to 6 carbon atoms. Examples include ethynylene (—C≡C—), propynylene (—CH₂—C≡C—), and the like.

“Acyl” refers to groups H-C(O)—, alkyl-C(O)—, cycloalkyl-C(O)—, aryl-C(O)—, heteroaryl-C(O)— and heterocyclyl-C(O)—, where alkyl, cycloalkyl, aryl, heteroaryl and heterocyclyl are as described herein.

“Oxyacyl” refers to groups HOC(O)—, alkyl-OC(O)—, cycloalkyl-OC(O)—, aryl-OC(O)—, heteroaryl-OC(O)—, and heterocyclyl-OC(O)—, where alkyl, cycloalkyl, aryl, heteroaryl and heterocyclyl are as described herein.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminoacyl” refers to the group —C(O)NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Acylamino” refers to the group —NR″C(O)R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyloxy” refers to the groups —OC(O)-alkyl, —OC(O)-aryl, —C(O)O-heteroaryl, and —C(O)O-heterocyclyl where alkyl, aryl, heteroaryl and heterocyclyl are as described herein.

“Aminoacyloxy” refers to the groups —OC(O)NR″-alkyl, —OC(O)NR″-aryl, —OC(O)NR″-heteroaryl, and —OC(O)NR″-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacylamino” refers to the groups —NR″C(O)O-alkyl, —NR″C(O)O-aryl, —NR″C(O)O-heteroaryl, and NR″C(O)O-heterocyclyl where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxyacyloxy” refers to the groups —OC(O)O-alkyl, —O—C(O)O-aryl, —OC(O)O-heteroaryl, and —OC(O)O-heterocyclyl where alkyl, cycloalkyl, aryl, heteroaryl, and heterocyclyl are as described herein.

“Acylimino” refers to the groups —C(NR″)—R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyliminoxy” refers to the groups —O—C(NR″)—R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Oxyacylimino” refers to the groups —C(NR″)—OR″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, indanyl, 1,2,3,4-tetrahydronapthalenyl and the like.

“Cycloalkenyl” refers to cyclic alkenyl groups having a single cyclic ring or multiple condensed rings, and at least one point of internal unsaturation, preferably incorporating 4 to 11 carbon atoms. Examples of suitable cycloalkenyl groups include, for instance, cyclobut-2-enyl, cyclopent-3-enyl, cyclohex-4-enyl, cyclooct-3-enyl, indenyl and the like.

“Halo” or “halogen” refers to fluoro, chloro, bromo and iodo.

“Heteroaryl” refers to a monovalent aromatic heterocyclic group which fulfils the Hückel criteria for aromaticity (i.e., contains 4n+2π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (e.g., pyridyl, pyrrolyl or N-oxides thereof or furyl) or multiple condensed rings (e.g., indolizinyl, benzoimidazolyl, coumarinyl, quinolinyl, isoquinolinyl or benzothienyl). It will be understood that where, for instance, R₂ or R′ is an optionally substituted heteroaryl which has one or more ring heteroatoms, the heteroaryl group can be connected to the core molecule of the compounds of the present invention, through a C—C or C-heteroatom bond, in particular a C—N bond.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Heteroarylene” refers to a divalent heteroaryl group wherein the heteroaryl group is as described above.

“Heterocyclylene” refers to a divalent heterocyclyl group wherein the heterocyclyl group is as described above.

“Thio” refers to groups H—S—alkyl-S—, cycloalkyl-S—, aryl-S—, heteroaryl-S—, and heterocyclyl-S—, where alkyl, cycloalkyl, aryl, heteroaryl and heterocyclyl are as described herein.

“Thioacyl” refers to groups H—C(S)—, alkyl-C(S)—, cycloalkyl-C(S)—, aryl-C(S)—, heteroaryl-C(S)—, and heterocyclyl-C(S)—, where alkyl, cycloalkyl, aryl, heteroaryl and heterocyclyl are as described herein.

“Oxythioacyl” refers to groups HO—C(S)—, alkylO-(S)—, cycloalkylO-C(S)—, arylO-C(S)—, heteroarylO-C(S)—, and heterocyclylO-C(S)—, where alkyl, cycloalkyl, aryl, heteroaryl and heterocyclyl are as described herein.

“Oxythioacyloxy” refers to groups HO—C(S)—O—, alkylO-C(S)—O—cycloalkylO-C(S)—O—arylO-C(S)—O—heteroarylO-C(S)—O—and heterocyclylO-C(S)—O—where alkyl, cycloalkyl, aryl, heteroaryl and heterocyclyl are as described herein.

“Phosphorylamino” refers to the groups —NR″—P(O)(R″′)(OR″″) where R″ represents H, alkyl, cycloalkyl, alkenyl, or aryl, R″′ represents OR″″ or is hydroxy or amino and R″″ is alkyl, cycloalkyl, aryl or arylalkyl, where alkyl, amino, alkenyl, aryl, cycloalkyl, and arylalkyl are as described herein.

“Thioacyloxy” refers to groups H—C(S)—O—, alkyl-C(S)—O—, cycloalkyl-C(S)—O—, aryl-C(S)—O—, heteroaryl-C(S)—O—, and heterocyclyl-C(S)—O—, where alkyl, cycloalkyl, aryl, heteroaryl, and heterocyclyl are as described herein.

“Sulfinyl” refers to groups H—S(O)—, alkyl-S(O)—, cycloalkyl-S(O)—, aryl-S(O)—, heteroaryl-S(O)—, and heterocyclyl-S(O)—, where alkyl, cycloalkyl, aryl, heteroaryl and heterocyclyl are as described herein.

“Sulfonyl” refers to groups H—S(O)₂—, alkyl-S(O)₂—, cycloalkyl-S(O)₂—, aryl-S(O)₂—, heteroaryl-S(O)₂—, and heterocyclyl-S(O)₂—, where alkyl, cycloalkyl, aryl, heteroaryl and heterocyclyl are as described herein.

“Sulfinylamino” refers to groups H—S(O)—NR″—, alkyl-S(O)—NR″—, cycloalkyl-S(O)—NR″—, aryl-S(O)—NR″—, heteroaryl-S(O)—NR″—, and heterocyclyl-S(O)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Sulfonylamino” refers to groups H—S(O)₂—NR″—, alkyl-S(O)₂—NR″—, cycloalkyl-S(O)₂—NR″—, aryl-S(O)₂—NR″—, heteroaryl-S(O)₂—NR″—, and heterocyclyl-S(O)₂—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxysulfinylamino” refers to groups HO—S(O)—NR″—, alkylO-S(O)—NR″—, cycloalkylO-S(O)—NR″—, arylO-S(O)—NR″—, heteroarylO-S(O)—NR″—, and heterocyclylO-S(O)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Oxysulfonylamino” refers to groups HO—S(O)₂—NR″—, alkylO-S(O)₂—NR″—, cycloalkylO-S(O)₂—NR″—, arylO-S(O)₂—NR″—, heteroarylO-S(O)₂—NR″—, and heterocyclylO-S(O)₂—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminothioacyl” refers to groups R″R″N—C(S)—, where each R″ is independently hydrogen, alkyl, cycloalkyl, aryl, heteroaryl, and heterocyclic and where each of alkyl, cycloalkyl, aryl, heteroaryl and heterocyclyl is as described herein.

“Thioacylamino” refers to groups H—C(S)—NR″—, alkyl-C(S)—NR″—, cycloalkyl-C(S)—NR″—, aryl-C(S)—NR″—, heteroaryl-C(S)—NR″—, and heterocyclyl-C(S)—NR″—, where R″ is independently hydrogen, alkyl, cycloalkyl, aryl, heteroaryl, and heterocyclyl and where each of alkyl, cycloalkyl, aryl, heteroaryl and heterocyclyl is as described herein.

“Aminosulfinyl” refers to groups R″R″N—S(O)—, where each R″ is independently hydrogen, alkyl, cycloalkyl, aryl, heteroaryl, and heterocyclic and where each of alkyl, cycloalkyl, aryl, heteroaryl and heterocyclyl is as described herein.

“Aminosulfonyl” refers to groups R″R″N—S(O)₂—, where each R″ is independently hydrogen, alkyl, cycloalkyl, aryl, heteroaryl, and heterocyclic and where each of alkyl, cycloalkyl, aryl, heteroaryl and heterocyclyl is as described herein.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroaryloxy, heterocyclyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono-and di-(substituted alkyl)amino, mono-and di-arylamino, mono- and di-heteroarylamino, mono- and di-heterocyclyl amino, and unsymmetric di-substituted amines having different substituents selected from alkyl, aryl, heteroaryl and heterocyclyl, and the like. For instance, an “optionally substituted amino” group may include amino acid and peptide residues.

In certain embodiments, the term “optionally substituted” is taken to mean that the groups may be substituted from 1 to 3 times independently selected from the groups consisting of oxo/hydroxy, halogen (in particular Cl, Br, F), C₁₋₆ alkyl, C₁₋₆ alkoxy, C₂₋₆ alkenyl, C₂₋₆ alkynyl, C₁₋₆ haloalkyl (in particular —CF₃), C₁₋₆ haloalkoxy (such as —OCF₃), C₂₋₆ alkenyloxy, C₂₋₆ alkynyloxy, arylalkyl (wherein alkyl is C₁₋₆), arylalkoxy (wherein alkyl is C₁₋₆), aryl, cyano, nitro, heteroaryl, C₁₋₆ heteroarylalkyl (wherein alkyl is C₁₋₆), heteroaryloxy, heterocyclyl, heterocyclylalkyl (wherein alkyl is C₁₋₆), heterocyclyloxy, oxyacyl, trialkylsilyl, trifluoromethanethio, trifluoroethenyl, amino, mono- and di-alkylamino, mono-and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl.

In other embodiments, the term “optionally substituted” is taken to mean that the groups may be substituted from 1 to 3 times independently selected from the groups consisting of hydroxy, halogen (in particular Cl, Br, F), C₁₋₆ alkyl, C₁₋₆ alkoxy, C₂₋₆ alkenyl, C₁₋₆ haloalkyl (in particular —CF₃), C₁₋₆ haloalkoxy (such as —OCF₃), arylalkyl (wherein alkyl is C₁₋₆), arylalkoxy (wherein alkyl is C₁₋₆), aryl, cyano, nitro, heteroaryl, trialkylsilyl, amino, mono- and di-alkylamino, mono-and di-(substituted alkyl)amino, and mono- and diarylamino.

In still further embodiments, the term “optionally substituted” is taken to mean that the groups may be substituted from 1 to 3 times independently selected from the groups consisting of hydroxy, halogen (in particular Cl, Br, F), hydroxethyl, hydroxpropyl, methyl, methoxy, cyano, pyridinyl, pyridinylmethyl, pyrazinyl, methylphenyl, benzyl, trimethylsilyl, phenyl, methylpyrazoyl, dimethylamino, fluorophenyl, tert-butyloxycarbonyl, amino or morpholinyl.

In certain embodiments, the present invention may be used in the treatment of a variety of diseases, disorders or conditions which would benefit from the negative modulation of α7 nAChR, including the step of administering to a patient in need of an effective amount of said compound or a pharmaceutically acceptable salt, solvate, prodrug, stereoisomer or tautomer thereof.

Such diseases, disorders or conditions include:
1) anxiety disorders, such as panic disorder with or without agoraphobia, agoraphobia without history of panic disorder, animal and other phobias including social phobias, obsessive-compulsive disorder, stress disorders including post-traumatic and acute stress disorder, and generalized or substance-induced anxiety disorder;
2) depressive or bipolar disorders, for example single-episode or recurrent major depressive disorder, dysthymic disorder, bipolar I and bipolar II manic disorders, and cyclothymic disorder;
3) anxiety and/or depression associated with psychotic disorders including schizophrenia;
4) anxiety and/or depression associated with neuroses;
5) anxiety and/or depression associated with convulsions;
6) anxiety and/or depression associated with migraine;
7) anxiety and/or depression associated with neurodegeneration arising from cerebral ischemia;
8) anxiety and/or depression associated with attention deficit hyperactivity disorder;
9) anxiety and/or depression associated with Tourette's syndrome;
10) anxiety and/or depression associated with speech disorders, including stuttering;
11) anxiety and/or depression associated with disorders of circadian rhythm, e.g., in subjects suffering from the effects of jet lag or shift work;
12) anxiety and/or depression associated with autism spectrum disorder (ASD); and
13) stress related disorders.

Further diseases, disorders or conditions for which compounds of the invention may be of benefit include:
1) anxiety and/or depression associated with pain and nociception;
2) anxiety and/or depression associated with emesis, including acute, delayed and anticipatory emesis, in particular emesis induced by chemotherapy or radiation, as well as motion sickness, and post-operative nausea and vomiting;
3) anxiety and/or depression associated with eating disorders including anorexia nervosa and bulimia nervosa;
4) anxiety and/or depression associated with premenstrual syndrome;
5) anxiety and/or depression associated with muscle spasm or spasticity, e.g., in paraplegic patients;
6) anxiety and/or depression associated with hearing disorders, including tinnitus and age-related hearing impairment;
7) anxiety and/or depression associated with urinary incontinence; and
8) anxiety and/or depression associated with the effects of substance abuse or dependency, including alcohol withdrawal.

Compounds of the present invention may be beneficial as pre-medication prior to anaesthesia or minor procedures such as endoscopy, including gastric endoscopy.

The compounds of the invention may be particularly useful in combination therapy, e.g., combining the treatment with other chemotherapeutic treatments (e.g., muscle relaxants, anticonvulants, hypnotics, anaesthetics, analgesics, antidepressants, antipsychotics, or other anxiolytics, etc.).

It will be understood that the compounds of the invention can be used in the treatment of any disease, disorder or condition which may be ameliorated by negative modulation of α7 nAChR.

With reference to formula (I), in an embodiment, L is a single bond.

With reference to formula (I) and sub-formulae (Ia), (Ib), and (Ic) in certain embodiments, R₁ may be selected from phenyl, oxazolyl, pyrrolyl, imidazolyl, pyrazolyl, pyridinyl, pyrazinyl, pyrimidinyl, pyridazinyl, indolizinyl, isoindolyl, indolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, isothiazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiadiazolyl, oxadiazolyl, oxatriazolyl, tetrazolyl, thiazolidinyl, thiophenyl, benzo[b]thiophenyl, morpholinyl, piperidinyl, pyrrolidinyl, tetrahydrofuranyl, or triazolyl, wherein any one of these groups may be optionally substituted with one to three substituents independently selected from hydroxy, halogen (in particular Cl, Br, F), C₁₋₆ alkyl, C₁₋₆ alkoxy, C₂₋₆ alkenyl, C₂₋₆ alkynyl, C₁₋₆ haloalkyl (such as —CF₃), C₁₋₆ haloalkoxy (such as —OCF₃), C₂₋₆ alkenyloxy, C₂₋₆ alkynyloxy, arylalkyl (wherein alkyl is C₁₋₆), arylalkoxy (wherein alkyl is C₁₋₆ ), aryl, cyano, nitro, heteroaryl, C₁₋₆ heteroarylalkyl (wherein alkyl is C₁₋₆), heteroaryloxy, heterocyclyl, heterocyclylalkyl (wherein alkyl is C₁₋₆), heterocyclyloxy, acyl, oxyacyl, trialkylsilyl, trifluoromethanethio, amino, mono- and di(C₁-C₆)-alkylamino, mono-and di-(substituted alkyl)amino, mono- and di-arylamino, mono- and di-heteroarylamino, mono-and di-heterocyclyl amino (including unsymmetric di-substituted amines having different substituents selected from alkyl, aryl, heteroaryl and heterocyclyl).

With reference to formula (I) and sub-formulae (Ia) and (Ib),

R₂ in some embodiments may be selected from H, optionally substituted C₁-C₃ alkyl, or optionally substituted C₃-C₆ heterocyclyl.

With reference to formula (I) and sub-formulae (Ia) and (Ib),

R₂ in some embodiments may be selected from H, or optionally substituted C₁-C₃ alkyl.

With reference to formula (I) and sub-formula (Ia), m may be 1 to 3 and R₃ and R₄ may be independently selected from H, hydroxyl, optionally substituted C₁-C₆ alkyl, optionally substituted C₁-C₆ alkoxyl, optionally substituted C₂-C₆ alkenyl, or optionally substituted C₂-C₆ alkynyl.

With reference to formula (I) and sub-formula (Ia), m may be 1 to 3 and R₃ and R₄ may be independently selected from H, hydroxyl, optionally substituted C₁-C₆ alkyl.

In an embodiment and with reference to sub-formula (Ib), m′ may be 1 and R₃ and R₄ may be independently selected from H, hydroxyl, optionally substituted C₁-C₆ alkyl, optionally substituted C₁-C₆ alkoxyl, optionally substituted C₂-C₆ alkenyl, or optionally substituted C₂-C₆ alkynyl.

In an embodiment and with reference to sub-formula (Ib), m′ may be 1 and R₃ and R₄ may be independently selected from H, hydroxyl, optionally substituted C₁-C₆ alkyl.

In an embodiment and with reference to sub-formula (Ib), m′ is 0.

In some embodiments, R₃ and R₄ is H and H, or methyl and methyl.

In some embodiments m is 1 or 2 and, R₃ and R₄ are both H; H, methyl; or methyl, methyl.

With reference to sub-formula (Ib), Y is selected from optionally substituted C₃-C₆ cycloalkyl, optionally substituted C₃-C₆ heterocyclyl, or optionally substituted C₃-C₆ cycloalkenyl. In an embodiment Y is cyclopropyl, cyclobutyl, cyclopentyl or cyclohexyl.

In certain embodiments Y (and with reference to sub formula I(b) is selected from cyclopropyl, cyclobutyl, cyclopentyl or cyclohexyl, and L is a single bond.

In certain embodiments Y (and with reference to sub formula I(b) is selected from cyclopropyl, cyclobutyl, cyclopentyl or cyclohexyl, L is a single bond and m′ is 1.

In certain embodiments Y (and with reference to sub formula I(b) is selected from cyclopropyl, cyclobutyl, cyclopentyl or cyclohexyl, L is a single bond and m′ is 0.

In certain embodiments Y (and with reference to sub formula I(b) is selected from cyclopropyl, cyclobutyl, cyclopentyl or cyclohexyl, L is a single bond, m′ is 1, R₃ and R₄ are both H.

In certain embodiments Y (and with reference to sub formula (Ib) is selected from cyclopropyl, cyclobutyl, cyclopentyl or cyclohexyl, L is a single bond, m′ is 0 and R₃ and R₄ are both H.

In certain embodiments and with reference to sub-formula (Ic) Z is selected from optionally substituted C₅-C₁₀ N-containing heterocyclyl, for instance piperidinyl and piperazinyl.

In certain embodiments and with reference to sub-formula (Id) T is an optionally substituted benzofused C₅-C₁₀ N-containing heterocyclyl.

In certain embodiments, the invention provides compounds of formula (II) or a pharmaceutically acceptable salt, solvate, prodrug, stereoisomer or tautomer thereof:

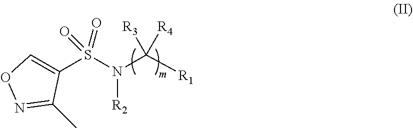

wherein R₁ is selected from optionally substituted heterocyclyl, optionally substituted aryl, or optionally substituted 6-membered heteroaryl; and
R₂ is selected from H, optionally substituted alkyl, optionally substituted aryl, optionally substituted cycloalkenyl, optionally substituted cycloalkyl, optionally substituted heteroaryl, optionally substituted heterocyclyl, optionally substituted acyl, or optionally substituted oxyacyl; and
R₃ and R₄ are independently selected from H and optionally substituted C₁-C₃ alkyl; and
m is an integer selected from 2 to 5.

In an embodiment m is 2 or 3.

In certain embodiments, the present invention relates to compounds of formula (II) which are represented by formula (IIa) and (IIb):

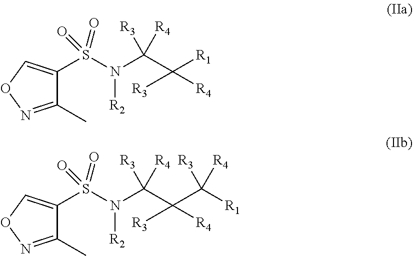

wherein R₁, R₂, R₃, and R₄ are as defined above for compounds of formula (II).

In certain embodiments, the present invention provides compounds of formula (II) which are represented by formula (IIa).

In certain embodiments, the present invention provides compounds of formula (II) which are represented by formula (IIb).

In a further embodiment and with reference to compound of formula (II) and sub formulae R₃ and R₄ are independently selected from H and H, or methyl and methyl.

In a further embodiment and with reference to compound of formula (II) and sub formulae R₁ is independently selected from optionally substituted phenyl, optionally substituted isoxazolyl, optionally substituted pyridyl, and optionally substituted pyrazolyl.

In other embodiments, the invention provides compounds of formula (III) or pharmaceutically acceptable salts, solvates, prodrugs, stereoisomers or tautomers thereof:

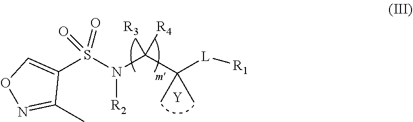

wherein R₁ is selected from optionally substituted heterocyclyl, optionally substituted aryl, or optionally substituted 6-memberd heteroaryl;
R₂ is selected from H, optionally substituted alkyl, optionally substituted aryl, optionally substituted cycloalkenyl, optionally substituted cycloalkyl, optionally substituted heteroaryl, optionally substituted heterocyclyl, optionally substituted acyl, or optionally substituted oxyacyl;
Y is selected from optionally substituted cycloalkyl, optionally substituted heterocyclyl, or optionally substituted cycloalkenyl.
L is a single bond;
R₃ and R₄, if present, are independently H or C₁-C₃ alkyl; and
m′ is 0 or 1.

In other embodiments the invention provides compounds of formula (IV) or a pharmaceutically acceptable salt, solvate, prodrug, stereoisomer or tautomer thereof:

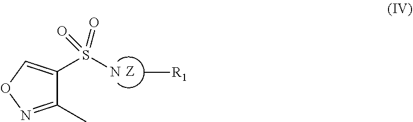

wherein R₁ is selected from optionally substituted heterocyclyl, optionally substituted aryl, optionally substituted 6-membered heteroaryl; and
Z is optionally substituted C₅-C₁₀ N-heterocyclyl.

In certain embodiments, Z—R₁ is substituted C₅-C₁₀ N-heterocyclyl.

In further embodiments, Z—R₁ is selected from:

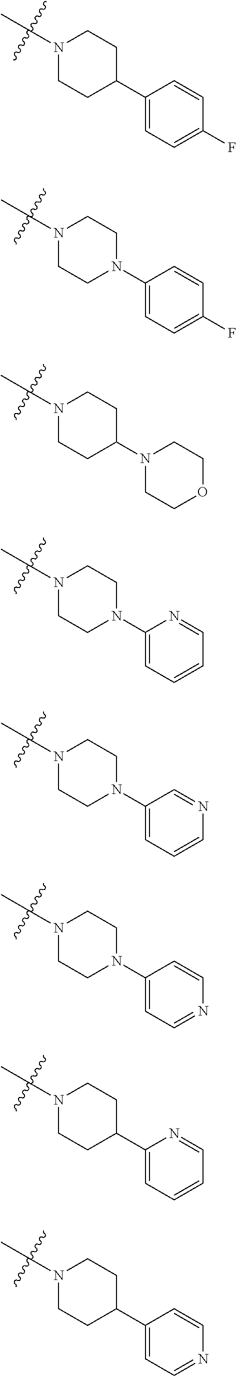

In certain other embodiments the invention provides compounds of formula (V) or pharmaceutically acceptable salts, solvates, prodrugs, stereoisomers or tautomers thereof:

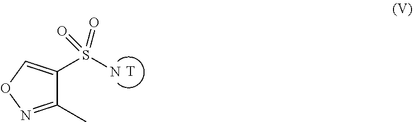

wherein T is a benzofused C₅-C₁₀ N-containing heterocyclyl.

In certain embodiments T is selected from:

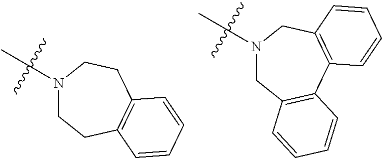 [SMILES: *N1CCc2ccccc2CC1 ; *N1Cc2ccccc2-c2ccccc2C1]

In the list below (which are representative examples of compounds of the present invention) the structures contain one or more stereogenic centers, the respective structures are depicted in an arbitrary absolute configuration. These structures also include the respective structure such as salts, tautomers, opposite stereoisomers as well as mixtures of isomers in all ratios including racemates:

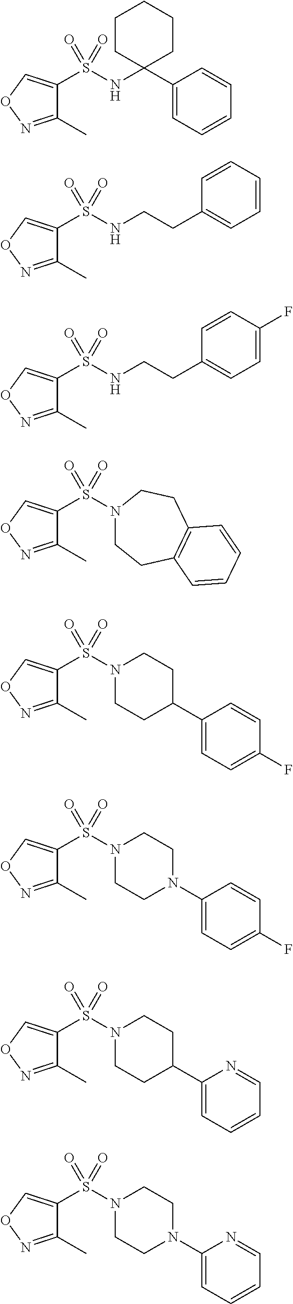

The preparation of sulonamides as starting materials in the above synthetic procedures may be accomplished using conventional chemistry (see for instance, D. T. Davies, Aromatic Heterocyclic Chemistry, 1993, Oxford Press, New York). Many such starting compounds have also been reported in the literature.

Other compounds of formulae can be prepared by the addition, removal or modification of existing substituents. This could be achieved by using standard techniques for functional group inter-conversion that are well known in the industry, such as those described in “Comprehensive organic transformations: a guide to functional group preparations” by Larock R. C., New York, VCH Publishers, Inc. 1989.

Examples of functional group inter-conversions are: —C(O)NR*R** from —CO₂CH₃ by heating with or without catalytic metal cyanide, e.g., NaCN, and HNR*R** in CH₃OH; —OC(O)R from —OH with e.g., ClC(O)R in pyridine; —NC(S)NR*R** from —NHR with an alkylisothiocyanate or thiocyanic acid; —NRC(O)OR* from —NHR with alkyl chloroformate; —NRC(O)NR*R** from —NHR by treatment with an isocyanate, e.g., HN═C═O or RN═C═O; —NRC(O)R* from —NHR by treatment with ClC(O)R* in pyridine; —C(═NR)NR*R** from —C(NR*R**)SR with H₃NR⁺OAc⁻ by heating in alcohol; —C(NR*R**)SR from —C(S)NR*R** with R—I in an inert solvent, e.g., acetone; —C(S)NR*R** (where R* or R** is not hydrogen) from —C(S)NH₂ with HNR*R**; —C(═NCN)—NR*R** from —C(═NR*R**)—SR with NH₂CN by heating in anhydrous alcohol, alternatively from —C(=NH)—NR*R** by treatment with BrCN and NaOEt in EtOH; —NR—C(═NCN)SR from —NHR* by treatment with (RS)₂C═NCN; —NR**SO₂R from —NHR* by treatment with ClSO₂R by heating in pyridine; —NR*C(S)R from —NR*C(O)R by treatment with Lawesson's reagent [2,4-bis(4-methoxyphenyl)-1,3,2,4-dithiadiphosphetane-2,4-disulfide]; —NRSO₂CF₃ from —NHR with triflic anhydride and base, —CH(NH₂)CHO from —CH(NH₂)C(O)OR* with Na(Hg) and HCl/EtOH; —CH₂C(O)OH from —C(O)OH by treatment with SOCl₂ then CH₂N₂ then H₂O/Ag₂O; —C(O)OH from —CH₂C(O)OCH₃ by treatment with PhMgX/HX then acetic anhydride then CrO₃; R—OC(O)R* from RC(O)R* by R**CO₃H; —CCH₂OH from —C(O)OR* with Na/R*OH; —CHCH₂ from —CH₂CH₂OH by the Chugaev reaction; —NH₂ from —C(O)OH by the Curtius reaction; —NH₂ from —C(O)NHOH with TsCl/base then H₂O; —CHC(O)CHR from —CHCHOHCHR by using the Dess-Martin Periodinane regent or CrO₃/aqH₂SO₄/acetone; —C₆H₅CHO from —C₆H₅CH₃ with CrO₂Cl₂; —CHO from —CN with SnCl₂/HCl; —CN from —C(O)NHR with PCl₅; —CH₂R from —C(O)R with N₂H₄/KOH.

During the reactions described above a number of the moieties may need to be protected. Suitable protecting groups are well known in industry and have been described in many references such as Protecting Groups in Organic Synthesis, Greene T W, Wiley-Interscience, New York, 1981.

The invention also provides for the use of compounds of formula (I), (II), (III), (IV) or (V) (and sub-formulae) or a pharmaceutically acceptable salt, solvate, prodrug, stereoisomer or tautomer thereof in the manufacture of medicaments for treating diseases, disorders or conditions which would benefit from the modulation of α7 nAChR.

There is also provided methods of treatment of diseases, disorders or conditions which would benefit from the modulation of α7 nAChR comprising the administration of an effective amount of at least one compound of formula (I), (II), (III), (IV) or (V) (and sub-formulae) or a pharmaceutically acceptable salt, solvate, prodrug, stereoisomer or tautomer thereof to a subject in need thereof.

The compounds of the invention may be particularly useful in combination therapy, e.g., combining the treatment with other chemotherapeutic treatments (e.g., muscle relaxants, anticonvulants, hypnotics, anaesthetics, analgesics or other anxiolytics, etc.).

It will be understood that the compounds of the invention can be used in the treatment of any disease state which may be ameliorated by negative modulation of the alpha 7 nicotinic receptor complex.

The compounds of the invention are administered to the subject in a treatment effective amount. As used herein, a treatment effective amount is intended to include at least partially attaining the desired effect, or delaying the onset of, or inhibiting the progression of, or halting or reversing altogether the onset or progression of the particular disease of condition being treated.

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages lie within the range of about 0.1 ng per kg of body weight to 1 g per kg of body weight per dosage. The dosage may be in the range of 1 mg to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another preferred embodiment, the dosage may be in the range of 1 mg to 100 mg per kg of body weight per dosage, such as up to 50 mg per body weight per dosage.

Suitable dosage amounts and dosing regimens can be determined by the attending physician and may depend on the particular condition being treated, the severity of the condition as well as the general age, health and weight of the subject.

The active ingredient may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the subject. Compositions include those suitable for oral, rectal, nasal, topical (including buccal and sublingual), vaginal or parental (including subcutaneous, intramuscular, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g., inert diluent, preservative disintegrant (e.g., sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

Compositions for rectal administration may be presented as a suppository with a suitable base comprising, for example, cocoa butter, glycerin, gelatine or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Preferably, the compounds of the present invention may be administered to a subject as a pharmaceutically acceptable salt. It will be appreciated however that non-pharmaceutically acceptable salts also fall within the scope of the present invention since these may be useful as intermediates in the preparation of pharmaceutically acceptable salts. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts e.g., sodium or potassium salts, or alkyl esters (e.g., methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any compound that is a prodrug of a compound of formula (I), (II), (III), (IV) or (V) (and sub-formulae) is also within the scope and spirit of the invention. The term “pro-drug” is used in its broadest sense and encompasses those derivatives that are converted in vivo to the compounds of the invention. Such derivatives would readily occur to those skilled in the art, and include, for example, compounds where a free hydroxy group (for instance at the CR₂ position) is converted into an ester, such as an acetate or phosphate ester, or where a free amino group is (for instance at the CR₂ position) converted into an amide (e.g., α-aminoacid amide). Procedures for esterifying, e.g., acylating, the compounds of the invention are well known in the art and may include treatment of the compound with an appropriate carboxylic acid, anhydride or chloride in the presence of a suitable catalyst or base. Any compound that is a prodrug of a compound of the invention is within the scope and spirit of the invention.

The compounds of the invention may be in crystalline form either as the free compounds or as solvates (e.g., hydrates) and it is intended that both forms are within the scope of the present invention. Methods of solvation are generally known within the art.

It will also be recognised that compounds of the invention may possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or mixtures may be resolved by conventional methods, e.g., chromatography, or use of a resolving agent.

Furthermore, depending on the substitution pattern the compounds of the present invention may be capable of undergoing tautomerism. Accordingly, all possible tautomers of a compound of the present invention fall within the scope and spirit of the invention.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES—Synthetic Procedure

All anhydrous solvents were commercially obtained and stored in Sure-Seal bottles under nitrogen. All other reagents and solvents were purchased as the highest grade available and used without further purification. Thin-layer chromatography (TLC) analysis of reaction mixtures was performed using Merck silica gel 60 F254 TLC plates and visualized using ultraviolet light. Silica gel 60 (40-63 μm, Merck) was used for flash chromatography. Preparative thin layer chromatography (PTLC) was performed using Merck silica gel 60 F254 PLC plates. SUPELCO Discovery® DSC-NH2 or DSC-SCX SPE columns were purchased from Sigma-Aldrich. Melting points were measured using an Electrothermal 1002 apparatus and were uncorrected. ¹H NMR spectra were obtained on a Bruker Advance 300 NMR (300 MHz) spectrometer or an Agilent DD2 (500 MHz) spectrometer using residual signal of deuterated NMR solvent as internal reference. Mass spectral data and purity of all compounds were acquired on an Agilent LCMS-Ion Trap-1200 Series. Mass spectra were obtained on an Agilent Ion Trap applying electrospray ionization (ESI). Purity of all compounds was obtained using a Nucleodur 3 μm 4.6×150 mm reverse-phase column. The eluent was a linear gradient with a flow rate of 1.3 mL/min from 95% A and 5% B to 5% A and 95% B in 8.5 min (solvent A, H₂O with 0.1% HCO₂H; solvent B, acetonitrile with 0.1% HCO₂H). The compounds were detected at their maximum of absorbance. Alternatively LCMS was performed using an Agilent Technologies 1260 Infinity, 6120 Quadrupole LC/MS and a Phenomenex Luna-C8 column 4.6×150 mm, 5μ, with mobile phase 80% ACN, 15% H₂O, 5% buffer (1L 3:1 MeOH/H₂O, 315 mg HCO₂NH₄, 1 mL AcOH) and MS detection (ESI method).

In the examples below, in case the structures contain one or more stereogenic centres, the respective structure is depicted in an arbitrary absolute configuration. These structures depict single enantiomers as well as mixtures of enantiomers in all ratios, and/or mixtures of diastereoisomers in all ratios.

### General Procedures

### General Procedure A: Sulfonamide formation

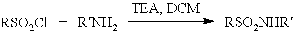

To a solution of amine (1.0 equiv.) in dichloromethane or tetrahydrofuran (0.1 M) were successively added triethylamine (1.5-3.0 equiv.) and sulfonyl chloride (1-1.5 equiv.). The reaction mixture was stirred at room temperature until completion. Work up of the reaction was either by Method A: addition of 1 M hydrochloric acid and chloroform followed by separation of the organic phase and removal of the solvent, or by Method B: the solvent was removed from the reaction mixture, or by Method C: addition of saturated sodium hydrogen carbonate followed by separation of the organic phase, drying over Na₂SO₄, and removal of the solvent. The crude obtained was purified by chromatography using the solvents indicated to furnish the pure coupling product.

### 3-Methyl-N-(1-phenylcyclohexyl)isoxazole-4-sulfonamide (1)

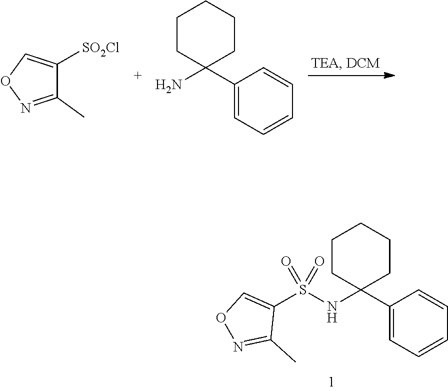

1-Phenylcyclohexyl-1-amine (73 mg, 0.415 mmol) and 3-methyl-1,2-oxazole-4-sulfonyl chloride (100 mg, 0.415 mmol) were reacted as described under General Procedure A (18 h at RT), worked up using Method A and the crude purified by PTLC using chloroform to furnish the title compound (30 mg, 23%).

¹H NMR (500 MHz, CDCl₃) δ 7.73 (s, 1H), 7.31-7.15 (m, 5H), 5.05 (s, 1H), 2.32 (s, 3H), 2.28-2.10 (m, 4H), 1.75-1.51 (m, 5H), 1.44-1.34 (m, 1H). ESIMS m/z [M−H]⁻ 319.1.

### 3-Methyl-N-phenethylisoxazole-4-sulfonamide (2)

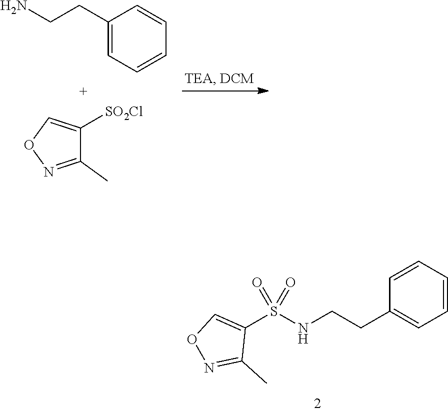

2-Phenylethylamine (66 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by PTLC using dichloromethane to furnish the title compound (117 mg, 78%).

¹H NMR (500 MHz, CDCl₃) δ 8.67 (s, 1H), 7.34-7.22 (m, 3H), 7.12 (d, J=7.2 Hz, 2H), 4.73 (s, br, 1H), 3.31 (app q, J=6.5 Hz, 2H), 2.79 (t, J=6.7 Hz, 2H), 2.26 (s, 3H). ESIMS m/z [M−H]⁻265.1.

### N-(4-Fluorophenethyl)-3-methylisoxazole-4-sulfonamide (3)

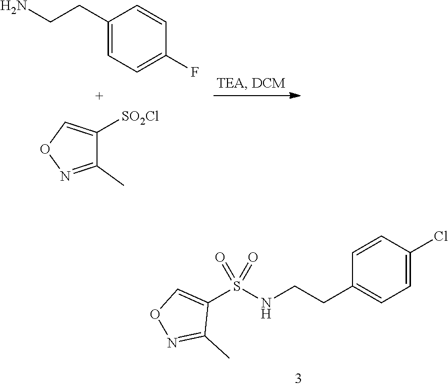

4-Fluorophenethylamine (77 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by PTLC using ethyl acetate/dichloromethane (1/9) to furnish the title compound (138 mg, 88%).

¹H NMR (500 MHz, CDCl₃) δ 8.72 (s, 1H), 7.14-7.07 (m, 2H), 7.00 (app t, J=8.6 Hz, 2H), 4.60 (s, br, 1H), 3.29 (app q, J=6.6 Hz, 2H), 2.78 (t, J=6.8 Hz, 2H), 2.32 (s, 3H). ESIMS m/z [M−H]⁻ 283.0.

### N-(2-(6-Chloropyridin-3-yl)ethyl)-3-methylisoxazole-4-sulfonamide (4)

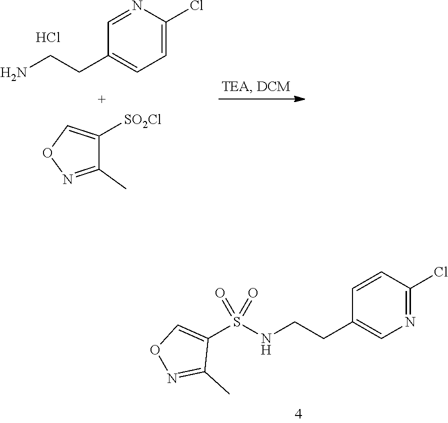

2-(Chloropyridin-3-yl)ethanamine hydrochloride (106 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by PTLC using ethyl acetate/dichloromethane (3/7) to furnish the title compound (81 mg, 49%).

¹H NMR (500 MHz, CDCl₃) δ 8.78 (s, 1H), 8.17 (d, J=2.0 Hz, 1H), 7.52 (dd, J=8.1, 2.0 Hz, 1H), 7.22 (d, J=8.1 Hz, 1H), 6.22 (br s, 1H), 3.34 (app q, J=6.4 Hz, 2H), 2.88 (t, J=6.5 Hz, 2H), 2.39 (s, 3H). ESIMS m/z [M−H]⁻ 300.0.

### 3-Methyl-N-(2-methyl-2-phenyl-propyl)isoxazole-4-sulfonamide (5)

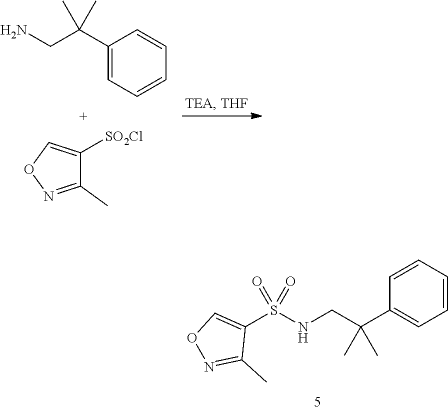

2-Methyl-2-phenylpropan-1-amine (49 mg, 0.33 mmol) and 3-methyl-1,2-oxazole-4-sulfonyl chloride (60 mg, 0.33 mmol) were reacted as described under General Procedure A (18 h at RT), worked up using Method C and the crude was purified by flash chromatography using cyclohexane/ethyl acetate (5/5) to furnish the title compound (59 mg, 60%).

¹H NMR (300 MHz, DMSO-d₆) δ 9.34 (s, 1H), 7.81 (t, J=6.3 Hz, 1H), 7.31-7.22 (m, 4H), 7.16 (t, J=6.4 Hz, 1H), 3.00 (d, J=6.4 Hz, 2H), 2.24 (s, 3H), 1.23 (s, 6H). ESIMS m/z [M+H]⁺ 295.2.

### 3-Methyl-N-[(1-phenylcyclopropyl)methyl]isoxazole-4-sulfonamide (6)

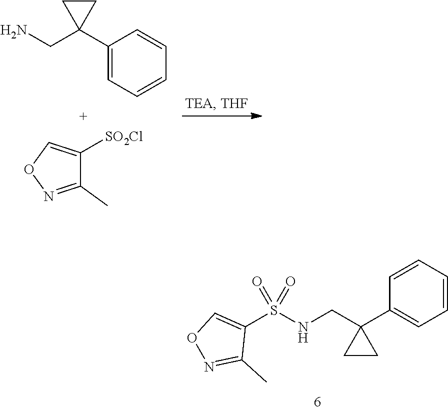

(1-Phenylcyclopropyl)methylamine (48 mg, 0.33 mmol) and 3-methyl-1,2-oxazole-4-sulfonyl chloride 3-methylisoxazole-4-sulfonyl chloride (60 mg, 0.33 mmol) were reacted as described under General Procedure A (18 h at RT), worked up using Method C and the crude was purified by flash chromatography using cyclohexane/ethyl acetate (5/5) to furnish the title compound (90 mg, 92%).

¹H NMR (300 MHz, DMSO-d₆) δ 9.30 (s, 1H), 8.05 (t, J=5.9 Hz, 1H), 7.25-7.12 (m, 5H), 3.09 (d, J=5.9 Hz, 2H), 2.19 (s, 3H), 0.85-0.82 (m, 2H), 0.75-0.71 (m, 2H). ESIMS m/z [M+H]⁺ 293.2.

### 4-(4-(4-Fluorophenyepiperidin-1-ylsulfonyl)-3-methylisoxazole (7)

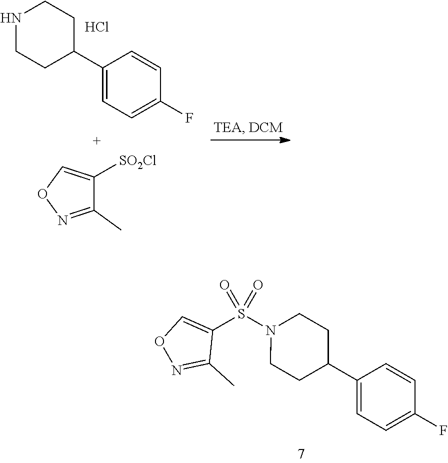

4-(4-Fluorophenyl)piperidine hydrochloride (118 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by flash chromatography using dichloromethane to furnish the title compound (120 mg, 67%).

¹H NMR (500 MHz, CDCl₃) δ 8.77 (s, 1H), 7.18-7.10 (m, 2H), 7.04-6.97 (d, 2H), 3.99-3.90 (m, 2H), 2.65-2.45 (m, 6H), 1.98-1.90 (m, 2H), 1.86-1.75 (m, 2H). ESIMS m/z [M+H]⁺ 325.1.

### 4-(4-(4-Fluorophenyl)piperazin-1-ylsulfonyl)-3-methylisoxazole (8)

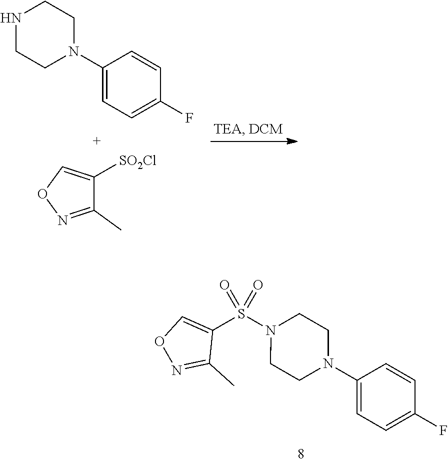

1-(4-Fluorophenyl)piperazine (99 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by flash chromatography using chloroform to furnish the title compound (146 mg, 81%).

¹H NMR (500 MHz, CDCl₃) δ 8.79 (s, 1H), 7.02-6.94 (m, 2H), 6.91-6.83 (m, 2H), 3.34-3.25 (m, 4H), 3.23-3.15 (m, 4H), 2.50 (s, 3H). ESIMS m/z [M+H]⁺ 326.1.

### 3-Methyl-4-(4-(pyridin-2-yppiperidin-1-ylsulfonyl)isoxazole (9)

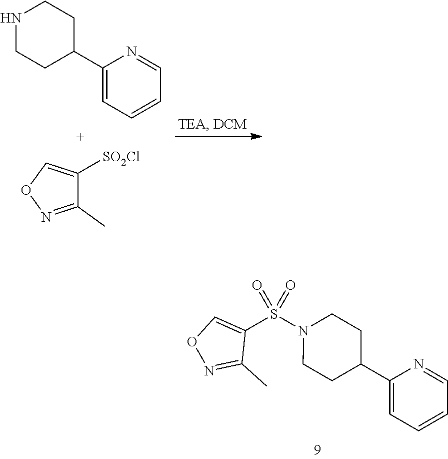

2-(Piperidin-4-yl)pyridine (89 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by PTLC using ethyl acetate/dichloromethane (2/8) to furnish the title compound (133 mg, 79%).

¹H NMR (500 MHz, CDCl₃) δ 8.77 (s, 1H), 8.54 (d, J=4.3 Hz, 1H), 7.64 (t, J=7.4 Hz, 1H), 7.19-7.11 (m, 2H), 3.99-3.89 (m, 2H), 2.80-2.63 (m, 3H), 2.49 (s, 3H), 2.11-2.03 (m, 2H), 2.00-1.88 (m, 2H). ESIMS m/z [M+H]⁺ 308.1.

### 3-Methyl-4-(4-(pyridin-2-yl)piperazin-1-ylsulfonyl)isoxazole (10)

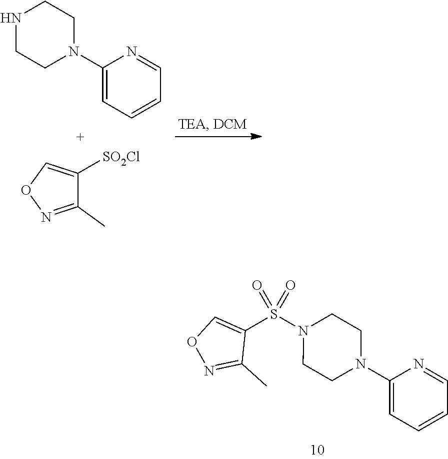

1-(2-Pyridyl)piperazine (90 mg, 0.551 mmol) and 3-methyl-1,2-oxazole-4-sulfonyl chloride (100 mg, 0.551 mmol) were reacted as described under General Procedure A (18 h at RT), worked up using Method B and the crude purified by flash chromatography using ethyl acetate/dichloromethane (1/9) to furnish the title compound (169 mg, 99%).

¹H NMR (500 MHz, CDCl₃) δ 8.77 (s, 1H), 8.19 (d, J=3.9 Hz, 1H), 7.51 (m, 1H), 6.69 (m, 1H), 6.64 (d, J=8.5 Hz, 1H), 3.70 (app t, J=4.9 Hz, 4H), 3.24 (app t, J=4.9 Hz, 4H), 2.49 (s, 3H). ESIMS m/z [M+H]⁺ 309.1.

### 3-Methyl-4-((1,2,4,5-tetrahydro-3H-benzo[d]azepin-3-yl)sulfonyl)isoxazole (11)

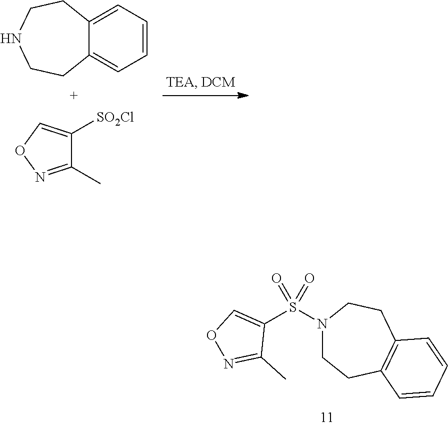

2,3,4,5-Tetrahydro-1H-benzo(d)azepine (81 mg, 0.551 mmol) and 3-methyl-1,2-oxoazole-4-sulfonyl chloride (100 mg, 0.551 mmol) were reacted as described under General Procedure A (18 h at RT), worked up using Method B and the crude was purified by PTLC using dichloromethane to furnish the title compound (138 mg, 86%).

¹H NMR (500 MHz, CDCl₃) δ 8.73 (s, 1H), 7.19-7.12 (m, 4H), 3.44-3.42 (m, 4H), 3.07-3.05 (m, 4H), 2.44 (s, 3H). ESIMS m/z [M+H]⁺ 293.1.

### Biological Data

### Screening of the Anxiolytic Effect

### Light/Dark Test

The light dark paradigm is based on a conflict between the innate aversion of rodents to brightly illuminated areas and on the spontaneous exploratory behaviour of the mice. If given a choice between a large brightly compartment versus a small dark compartment they spontaneously prefer the dark part. Anxiolytic compounds have been found to increase the number of entries into the bright compartment and the total time spent there. Anxiogenic compounds were observed to work in the opposite way.

The apparatus consists of two PVC (polyvinylchloride) boxes (19′ 19′ 15 cm) covered with Plexiglas. One of these boxes is darkened. The other box is illuminated by 100 W desk lamp placed 15 cm above and providing an illumination of about 4400 Lux. An opaque plastic tunnel (5′ 7′ 10 cm) separates the dark box from the illuminated one.

Animals were placed individually in the lit box, with head directed towards the tunnel. The time spent in the lit box and the number of transitions between the two boxes was recorded over a 5 min period after the first entry of the animal in the dark box. The total walked distance in the lit box was also recorded. Animals scored without entry into the lit box were excluded from the analysis.

### Test Compounds and Treatment

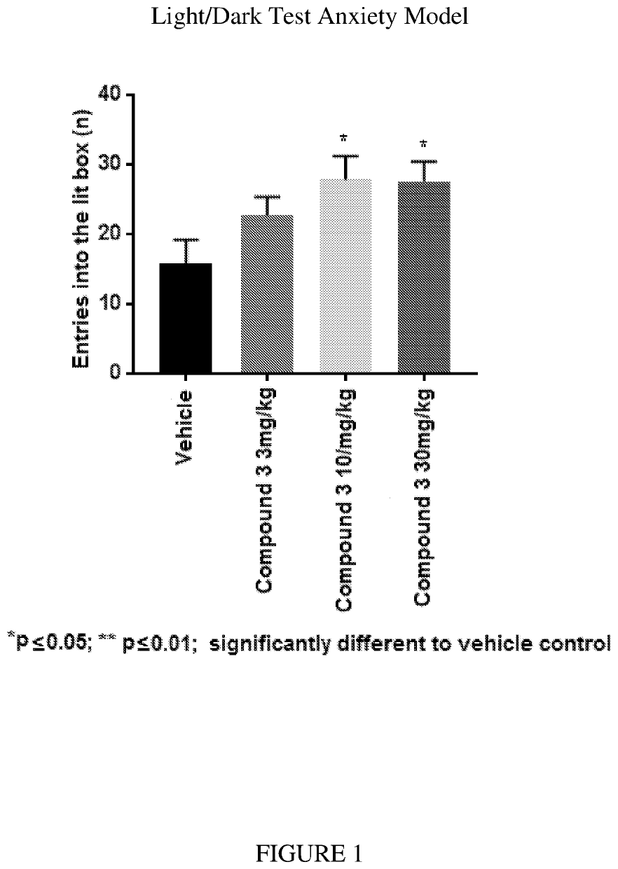

FIG. 1 illustrates the results of compound example 3 at three varying mg/kg dosage forms.

The test compound was prepared in 5% PEG400-0.9% NaCl.

It was administrated orally, 60 minutes before the implementation of the test.

Mean±sem of 10 mice.

### Compound Plate Preparation

Immediately prior to assay the supplied compound(s) were prepared in DMSO to concentrations that were 300× the final assay top concentration(s) of 10 μM, five dose-responses were prepared by 3-fold serial dilution in DMSO from the top concentration and aliquots were taken out from the respective concentrations and diluted 300× into external buffer to give the final assay concentrations. All wells included a final DMSO concentration of 0.33% including all control wells.

All compounds were fully soluble in external buffer by visual inspection.

| Ion Channel | EC₈₀ Control & Concentration |
|---|---|
| hnAChR α7/ric3 | 200 μM Ach |

### Electrophysiological Recording Solutions

The solutions for recording hnAChR α7/ric3 currents were:

| External Recording Solution | | Internal Recording Solution | | |
|---|---|---|---|---|
| NaCl | 137 | TRIS-PO4 | 110 | mM |
| KCl | 4 | TRIS-base | 28 | mM |
| MgCl₂ | 1 | CaCl₂ | 0.1 | mM |
| CaCl₂ | 1.8 | MaCl₂ | 2 | mM |
| HEPES | 10 | EGTA | 11 | mM |
| Glucose | 10 | MaATP | 4 | mM |

| | |
|---|---|
| pH 7.35 (titrated with NaOH) | pH 7.3 (titrated with TRIS-base) |

Experimental Protocols

All recordings were obtained from a holding potential of −60 mV.

Compounds were assayed using bump-in protocol. The compound addition sequence that was used for all additions was the same for all assays. One addition of the EC80 concentration of ACh (200 μM) was added to establish baseline response. The test compounds were tested on cells in increasing concentrations of 0.12, 0.37, 1.1, 3.3, 10 μM±200 μM ACh for all compounds. Each compound was applied for one minutes followed by the addition of 200 μM ACh for 1 second.

### Data Analysis

Peak inward currents in response to the ACh additions in the presence of increasing concentrations of compound were measured. All compound data have been normalized to the baseline peak current induced by addition of 200 μM ACh for 1 second:

Potentiation of ACh Response=(I^{Compound}^{⁺}^{ACh}/I^{ACh})

Where I (Compound+ACh) is the peak current induced by addition of 0.12, 0.37, 1.1, 3.3, 10 μM test compound+200 μM ACh after 1 minutes incubation of 0.12, 0.37, 1.1, 3.3, 10 μM test compound, I ACh is the baseline peak current induced by addition of 200 μM ACh. All data were first exported to an Excel compatible data file and then analyzed using Graph Pad Prism software.

### IonChannelProfiler Data Filters

| Data Filter | Platform | Criteria | |
|---|---|---|---|
| Rm | IonFlux HT | >60 | MΩ |
| Current Amplitude | IonFlux HT | >250 | pA |

### RESULTS

| | | IC50 |
|---|---|---|
| Compound | Structure | (uM) |
| 1 | 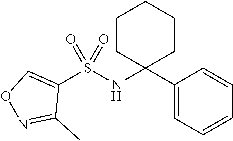 [SMILES: Cc1nocc1S(=O)(=O)NC1(c2ccccc2)CCCCC1] | 5.70 |
| 2 | 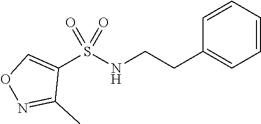 [SMILES: Cc1nocc1S(=O)(=O)NCCc1ccccc1] | 1.70 |
| 3 | 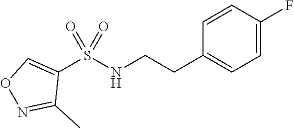 [SMILES: Cc1nocc1S(=O)(=O)NCCc1ccc(F)cc1] | 2.20 |
| 7 | 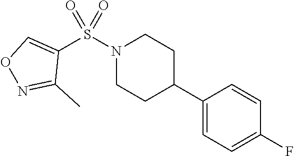 [SMILES: Cc1nocc1S(=O)(=O)N1CCC(c2ccc(F)cc2)CC1] | 0.40 |
| 8 | 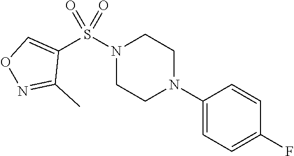 [SMILES: Cc1nocc1S(=O)(=O)N1CCN(c2ccc(F)cc2)CC1] | 2.20 |
| 10 | 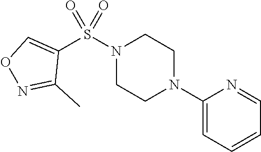 [SMILES: Cc1nocc1S(=O)(=O)N1CCN(c2ccccn2)CC1] | 6.50 |
| 11 | 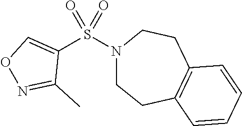 [SMILES: Cc1nocc1S(=O)(=O)N1CCc2ccccc2CC1] | 2.90 |

### BIBLIOGRAPHY

1. Barik J and Wonnacott S (2006) Indirect modulation by alpha7 nicotinic acetylcholine receptors of noradrenaline release in rat hippocampal slices: interaction with glutamate and GABA systems and effect of nicotine withdrawal. Mol Pharmacol. February; 69(2):618-28
2. Dickinson J A, et al. (2008) Presynaptic alpha 7- and beta 2-containing nicotinic acetylcholine receptors modulate excitatory amino acid release from rat prefrontal cortex nerve terminals via distinct cellular mechanisms. Mol Pharmacol. August; 74(2):348-59.
3. Livingstone P D, et al. (2009) Glutamate-dopamine crosstalk in the rat prefrontal cortex is modulated by Alpha7 nicotinic receptors and potentiated by PNU-120596 J Mol Neurosci. 2010 January; 40(1-2):172-6.
4. Quarta D, et al. (2009) Drug discrimination and neurochemical studies in alpha7 null mutant mice: tests for the role of nicotinic alpha7 receptors in dopamine release Psychopharmacology (Berl). 2009 April; 203(2):399-410
5. Janowsky D S, el-Yousef M K, Davis J M, Sekerke H J (1972) A cholinergic-adrenergic hypothesis of mania and depression. Lancet (7778):632-635.
6. Mitchell A J. (1998) The role of corticotropin releasing factor in depressive illness: a critical review. Neurosci Biobehav Rev; 22:635-651.29;
7. Nemeroff C B. 1998 The neurobiology of depression. Sci Am; 278:42-49
8. Saricicek A, et al. (2012) Persistent β2*-nicotinic acetylcholinergic receptor dysfunction in major depressive disorder. Am J Psychiatry 169(8):851-859.
9. Furey M L, Drevets W C (2006) Antidepressant efficacy of the antimuscarinic drug scopolamine: A randomized, placebo-controlled clinical trial. Arch Gen Psychiatry 63(10):1121-1129
10. Janowsky D S, Risch S C. Cholinomimetic and anticholinergic drugs used to investigate and acetylcholine hypothesis of affective disorders and stress. Drug Devel Res 1984; 4:125-142.
11. Mineur Y S, et al. (2013) Cholinergic signaling in the hippocampus regulates social stress resilience and anxiety- and depression-like behavior. PNAS 110(9):3573-3578.
12. Klein and Yakel 2006
13. Pidoplichko V I, et al. (2013) α7-Containing nicotinic acetylcholine receptors on interneurons of the basolateral amygdala and their role in the regulation of the network excitability. J Neurophysiol 110:2358-2369
14. Mineur Y S, et al. (2016) Multiple Nicotinic Acetylcholine Receptor Subtypes in the Mouse Amygdala Regulate Affective Behaviors and Response to Social Stress Neuropsychopharmacology 41(6):1579-87.

The claims defining the invention are as follows:

1. A compound of formula (I) or pharmaceutically acceptable salts thereof: 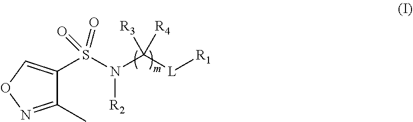
wherein R₁ is selected from optionally substituted heterocyclyl, optionally substituted aryl, optionally substituted 6-membered heteroaryl; and
R₂ is selected from H, optionally substituted alkyl, optionally substituted aryl, optionally substituted cycloalkenyl, optionally substituted cycloalkyl, optionally substituted heteroaryl, optionally substituted heterocyclyl, optionally substituted acyl, or optionally substituted oxyacyl; and
R₃ and R₄ are independently selected from H, hydroxyl, optionally substituted alkyl, optionally substituted alkoxyl, optionally substituted alkenyl, optionally substituted alkynyl, optionally substituted cycloalkyl, optionally substituted heterocyclyl, optionally substituted aryl, optionally substituted heteroaryl, or optionally substituted cycloalkenyl; or
R₃ and R₄ can be linked to form optionally substituted cycloalkyl, optionally substituted heterocyclyl, or optionally substituted cycloalkenyl; and
L is a single bond, N, CH, or a divalent linker selected from CH₂, optionally substituted C₃-C₆ cycloalkylene and optionally substituted C₃-C₆ cycloalkenylene; or
R₂ and L can be linked to form optionally substituted N-containing heterocyclyl where L is N or CH; or
R₂ and R₁ can be linked to form optionally substituted N-containing heterocyclyl where L is CH₂; and
m is an integer selected from 1 to 5,
excluding the following two compounds:

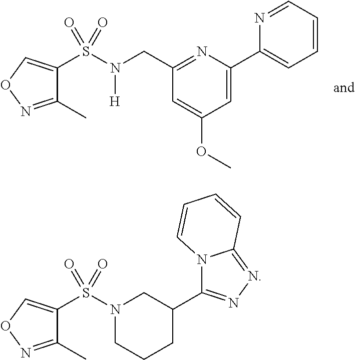

2. A compound of claim 1 or pharmaceutically acceptable salt, solvate, prodrug, stereoisomer or tautomer thereof selected from the sub-formulae: 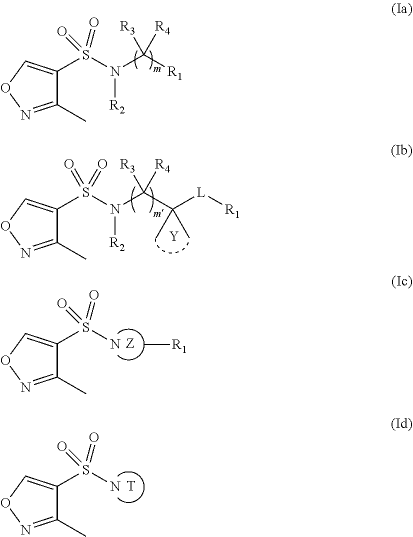
wherein R₁, R₂, R₃, R₄, L and m are as defined above in claim 1;
wherein Y is where one R₃ and R₄ is linked together to form optionally substituted cycloalkyl, optionally substituted heterocyclyl, or optionally substituted cycloalkenyl;
wherein Z is where R₂ and L is linked together to form optionally substituted N-containing heterocyclyl where L is N or CH;
wherein T is where R₂ and R₁ is linked together to form optionally substituted N-containing heterocyclyl where L is CH₂; and
m′ is 0 or 1.

3. A compound of claim 2, wherein the sub-formulae is selected from (Ia) or (Ib).

4. A compound of any one of claims 1 to 3, wherein R₁ is selected from phenyl, oxazolyl, pyrrolyl, imidazolyl, pyrazolyl, pyridinyl, pyrazinyl, pyrimidinyl, pyridazinyl, indolizinyl, isoindolyl, indolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, isothiazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7 -tetrahydrobenzo[b] thiophenyl, thiazolyl, thiadiazolyl, oxadiazolyl, oxatriazolyl, tetrazolyl, thiazolidinyl, thiophenyl, benzo[b]thiophenyl, morpholinyl, piperidinyl, pyrrolidinyl, tetrahydrofuranyl, or triazolyl,
wherein any one of these groups may be optionally substituted with one to three substituents independently selected from hydroxy, halogen (in particular Cl, Br, F), C₁₋₆ alkyl, C₁₋₆ alkoxy, C₂₋₆ alkenyl, C₂₋₆ alkynyl, C₁₋₆ haloalkyl (such as —CFA C₁₋₆ haloalkoxy (such as —OCF₃), C₂₋₆ alkenyloxy, C₂₋₆ alkynyloxy, arylalkyl (wherein alkyl is C₁₋₆), arylalkoxy (wherein alkyl is C₁₋₆), aryl, cyano, nitro, heteroaryl, C₁₋₆ heteroarylalkyl (wherein alkyl is C₁₋₆), heteroaryloxy, heterocyclyl, heterocyclylalkyl (wherein alkyl is C₁₋₆), heterocyclyloxy, acyl, oxyacyl, trialkylsilyl, trifluoromethanethio, amino, mono- and di(C₁-C₆)-alkylamino, mono-and di-(substituted alkyl)amino, mono- and di-arylamino, mono- and di-heteroarylamino, mono- and di-heterocyclyl amino (including unsymmetric di-substituted amines having different substituents selected from alkyl, aryl, heteroaryl and heterocyclyl).

5. A compound of claim 4, wherein R₁ is phenyl optionally substituted with one to three substituents independently selected from hydroxy, halogen (in particular Cl, Br, F), C₁₋₆ alkyl, C₁₋₆ alkoxy, C₂₋₆ alkenyl, C₂₋₆ alkynyl, C₁₋₆ haloalkyl (such as —CF₃), C₁₋₆ haloalkoxy (such as —OCF₃), C₂₋₆ alkenyloxy, C₂₋₆ alkynyloxy, arylalkyl (wherein alkyl is C₁₋₆), arylalkoxy (wherein alkyl is C₁₋₆), aryl, cyano, nitro, heteroaryl, C₁₋₆ heteroarylalkyl (wherein alkyl is C₁₋₆), heteroaryloxy, heterocyclyl, heterocyclylalkyl (wherein alkyl is C₁₋₆), heterocyclyloxy, acyl, oxyacyl, trialkylsilyl, trifluoromethanethio, amino, mono- and di(C₁-C₆)-alkylamino, mono-and di-(substituted alkyl)amino, mono- and di-arylamino, mono- and di-heteroarylamino, mono- and di-heterocyclyl amino (including unsymmetric di-substituted amines having different substituents selected from alkyl, aryl, heteroaryl and heterocyclyl).

6. A compound of claim 3, wherein R₂ is selected from H, optionally substituted C₁-C₃ alkyl, or optionally substituted C₃-C₆ heterocyclyl.

7. A compound of claim 6, wherein the sub-formulae is (Ia) and m is 1 to 3 and R₃ and R₄ is independently selected from H, hydroxyl, optionally substituted C₁-C₆ alkyl, optionally substituted C₁-C₆ alkoxyl, optionally substituted C₂-C₆ alkenyl, or optionally substituted C₂-C₆ alkynyl.

8. A compound of claim 7, wherein m is 1 to 3 and R₃ and R₄ is independently selected from H, hydroxyl, optionally substituted C₁-C₆ alkyl.

9. A compound of claim 3, wherein the sub-formulae is (Ib) and m′ is 0 or 1 and R₃ and R₄ is independently selected from H, hydroxyl, optionally substituted C₁-C₆ alkyl, optionally substituted C₁-C₆ alkoxyl, optionally substituted C₂-C₆ alkenyl, or optionally substituted C₂-C₆ alkynyl.

10. A compound of claim 9, wherein m′ is 1 and R₃ and R₄ is independently selected from H, hydroxyl, optionally substituted C₁-C₆ alkyl.

11. A compound of claim 9, wherein m′ is 0.

12. A compound of claim 1, wherein R₃ and R₄ is H and H, or methyl and methyl.

13. A compound of formula (II) or a pharmaceutically acceptable salt, solvate, prodrug, stereoisomer or tautomer thereof: 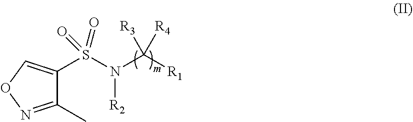
wherein R₁ is selected from optionally substituted heterocyclyl, optionally substituted aryl, or optionally substituted 6-membered heteroaryl; and
R₂ is selected from H, optionally substituted alkyl, optionally substituted aryl, optionally substituted cycloalkenyl, optionally substituted cycloalkyl, optionally substituted heteroaryl, optionally substituted heterocyclyl, optionally substituted acyl, or optionally substituted oxyacyl; and
R₃ and R₄ are independently selected from H and optionally substituted C₁-C₃ alkyl; and
m is an integer selected from 2 to 5.

14. A compound of claim 13, wherein m is 2 or 3.

15. A compound of claim 13 or 14 which are represented by formula (IIa) and (IIb): 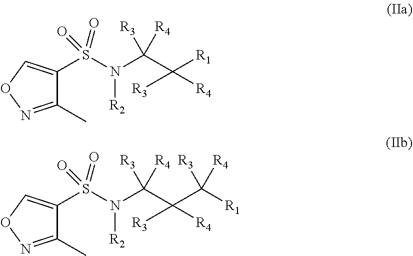
wherein R₁, R₂, R₃, and R₄ are as defined above for compounds of formula (II).

16. A compound of formula (III) or pharmaceutically acceptable salts, solvates, prodrugs, stereoisomers or tautomers thereof: 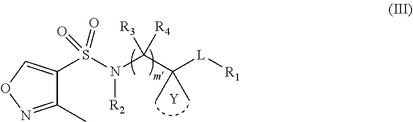
wherein R₁ is selected from optionally substituted heterocyclyl, optionally substituted aryl, or optionally substituted 6-memberd heteroaryl;
R₂ is selected from H, optionally substituted alkyl, optionally substituted aryl, optionally substituted cycloalkenyl, optionally substituted cycloalkyl, optionally substituted heteroaryl, optionally substituted heterocyclyl, optionally substituted acyl, or optionally substituted oxyacyl;
Y is selected from optionally substituted cycloalkyl, optionally substituted heterocyclyl, or optionally substituted cycloalkenyl.
L is a single bond;
R₃ and R₄, if present, are independently H or C₁-C₃ alkyl; and
m′ is 0 or 1.

17. A compound of formula (IV) or a pharmaceutically acceptable salt, solvate, prodrug, stereoisomer or tautomer thereof: 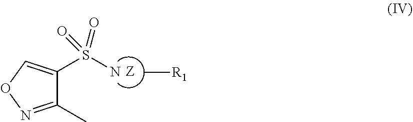
wherein R₁ is selected from optionally substituted heterocyclyl, optionally substituted aryl, optionally substituted 6-membered heteroaryl; and
Z is optionally substituted C₅-C₁₀ N-heterocyclyl.

18. A compound of formula (V) or pharmaceutically acceptable salts, solvates, prodrugs, stereoisomers or tautomers thereof: 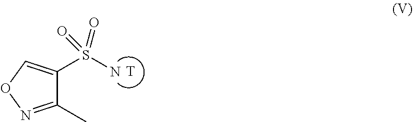
wherein T is a benzofused C₅-C₁₀ N-containing heterocyclyl.

19. A compound selected from:

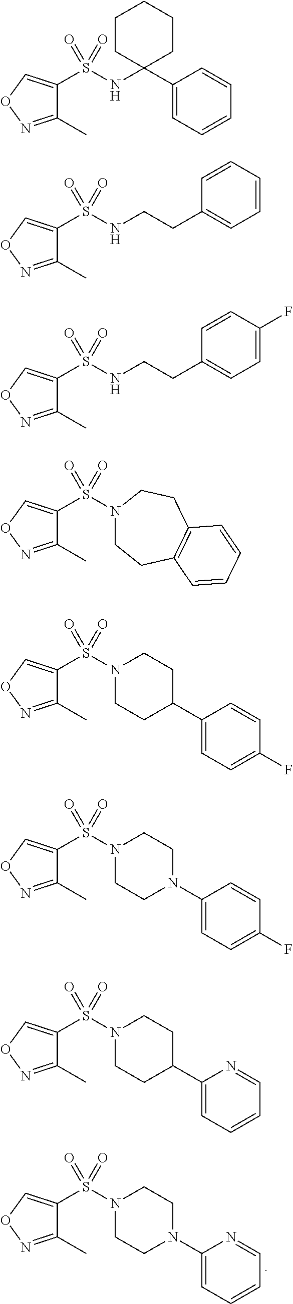

20. A method of medically treating a disease, disorder, or condition which would benefit from modulation of α7nAChR, said method including the step of administering an effective amount of a compound according to any one of claims 1 to 19 or a pharmaceutically acceptable salt thereof.

21. Use of a compound according to any one of claims 1 to 19 or pharmaceutically acceptable salts thereof, in the manufacture of a medicament for treating a disease, disorder or condition which would benefit from modulation of α7 nAChR.

22. Use of a compound according to any one of claims 1 to 19 or a pharmaceutical salt thereof, for treating a disease, disorder or condition which would benefit from modulation of α7 nAChR.

23. A method or use according to any one of claim 20, 21 or 22, wherein the disease, disorder or condition which would benefit from modulation of α7 nAChR is a disease, disorder or condition in which the benefit comes from the negative allosteric modulation of α7 nAChR.

24. A method or use according to claim 23 wherein the disease, disorder or condition in which the benefit comes from the negative allosteric modulation of α7 nAChR is a disease, disorder or condition selected from anxiety, depression, or a stress-related disorder.
